(19) United States
(12) Patent Application Publication
(10) Pub. No.: US 2009/0018809 A1
(43) Pub. Date: Jan. 15, 2009

(54) COMPUTER GENE

(75) Inventors: Karl-Heinz Zimmermann, Bayreuth (DE); Zoya Ignatova, Berlin (DE); Israel Marck Martinez-Perez, Baja California (MX)

Correspondence Address: PATENT CENTRAL LLC;Stephan A. Pendorf, 1401 Hollywood Boulevard, Hollywood, FL, 33020, US

(73) Assignees: Technische Universitaet Hamburg-Harburg (Technical University Hamburg-Hraburg), Hamburg (DE); Tutech Innovation GmbH, Hamburg (DE)

(21) Appl. No.: 12/280,593

(22) PCT Filed: Feb. 23, 2007
(86) PCT No.: PCT/EP07/01596

§ 371 Date: Aug. 25, 2008

(30) Foreign Application Priority Data
Feb. 23, 2006 (DE) 10 2006 009 000.4

(51) Int. Cl. G06F 9/455; C07H 21/00; C07H 1/00; C12P 19/34; A61K 31/7088; A61P 43/00

(52) U.S. Cl. 703/11; 536/22.1; 536/25.3; 435/91.1; 514/44

(57) ABSTRACT

The invention relates to the field of bioinformatics and in particular of biomolecular computing (‘DNA computing’). “Computational genes” comprising nucleic acids are provided which, via autonomous spontaneous self-assembly, can be produced in vivo by means of a biomolecular finite automaton.

The invention relates to a nucleic acid comprising at least one gene, a method of preparing same, a programmable biomolecular finite automaton and a composition.

The invention pertains to the field of bioinformatics and particularly biomolecular computing (“DNA computing”).

Already at the beginning of the 1960's, Feynman had the idea of performing massively parallel computations based on nanotechnology (R. P. Feynman: Miniaturization. In D. H. Gilberts (ed.), Reinhold, New York, 282-296, 1961). Adleman was then the first to find a solution to a small instance of the Hamiltonian path problem by a biomolecular computation in vitro with the aid of DNA molecules (Adleman, L., 1994, Molecular computing of solutions to combinatorial problems, Science, 266, 1021-1024).

In general, the biomolecular computation methods that became known since then require an intervention from outside. Among the most prominent models of the first generation are the sticker and the splicing model (T. Head: Formal language theory and DNA: An analysis of the generative capacity of specific recombinant behaviors. Bull. Math. Biology, 49, 737-759,1987; Roweis, S. E., Winfree, E., Burgoyne, R., Chelyapov, N. V., Goodman, M., Rothemund, R, Adleman, L.: A sticker based architecture for DNA computation. Proc. 2nd Ann. DIMACS, Princeton, 1-29, 1996). Both models are computationally complete and universal (L. Kari: DNA computing: arrival of biological mathematics. Math. Intell. 19, 9-22,1997; L. Kari, G. Paun, G. Rozenberg, A. Salomaa, and S. Yu: DNA computing, sticker systems, and universality. Acta Informatica, 35, 401-420, 1998). Based on these models a variety of DNA algorithms have been suggested to solve NP-hard problems. Such DNA algorithms are more efficient than in silico algorithms.

In the current models of biomolecular computing the computational processes are normally carried out autonomously. These computational processes happen by spontaneous self-assembly of smaller DNA molecules and are modulated by DNA manipulating enzymes. For example, nanostructures in form of periodic, two-dimensional lattices have been generated by small, branched DNA molecules (Winfree, E.: Algorithmic self-assembly of DNA. PhD Thesis, California Institute of Technology, 1998; E. Winfree, F. Liu, L. A. Wenzler and N. C. Seeman, Design and self-assembly of two-dimensional DNA Crystals. Nature, 394, 539-544, 1998; E. Winfree, X. Yang, N. C. Seeman, Universal computation via self-assembly of DNA: Some theory and experiments, Proc. 2nd Ann. DIMACS, 10-12, 1996). On such a two-dimensional lattice the design of an autonomous computationally universal turing machine is based (P. Yin, A. Turberfield, S. Sahu and J. H. Reif, Design of an autonomous DNA nano-mechanical device capable of universal computation and universal Translation Motion. Science, Adv. online publ., 2004). Further, several moving autonomous DNA structures were developed (Y. Chen, M. Wang and C. Mao: An autonomous DNA motor powered by a DNA enzyme. Angew. Int. Ed., 43, 2-5, 2004; J. H. Reif: The design of autonomous DNA nanomechanical devices. LNCS, 2568, 22-37, 2003; W. B. Sherman and N. C. Seeman: A precisely controlled DNA biped walking device. Nano. Lett., 2004; A. J. Turberfield, J. C. Mitchell, B. Yurke Jr., A. P. Mills, M. I. Blakey and F. C. Simmel: DNA fuel for free-running nanomachines. Phys. Rev. Lett., 90, 118102, 2003).

Further, an autonomous DNA model called “Shapiro model” has become known, that allows for the construction of finite automata with two input symbols and two states (Y. Benenson, T. Paz-Elizur, R. Adar, E. Keinan, Z. Livneh and E. Shapiro: Programmable and autonomous computing machine made of biomolecules. Nature, 414, 430-434, 2001; US patent application 20050075792). These automata, however, have a very small complexity (number of input symbols times number of states), whose increase is limited by the number of non-palindromic staggered ends (“sticky ends”). In addition, the DNA molecule coding the input is destroyed during the processing.

The Shapiro model has been expanded to stochastic finite automata. The probabilities of the transition rules are implemented by the relative molar concentrations of the corresponding DNA molecules (R. Adar, Y. Benenson, G. Linshiz, A. Rosner, N. Tishby and E. Shapiro: Stochastic computing with biomolecular automata. Proc. Nat. Acad. Sci. USA, 101, 9960-9965, 2004).

In addition, a model for the logical control of gene expression based upon the Shapiro model has been described (Y. Benenson, B. Gil, U. Ben-Dor, R. Adar and E. Shapiro: An autonomous molecular Computer for logical control of gene expression. Nature, Adv. online publ., 2004). This model uses biomolecules as input and biologically active molecules as output. The output molecules are single-stranded DNA molecules (ssDNA), which, however, are limited in their length (maximum 21 bp). This is due to the fact that the output molecule is embedded in the input molecule of this automaton in form of a hairpin structure and has to be protected against interaction with other molecules.

In eukaryotic organisms genes have a mosaic-like structure. The coding sequences of their genes can be interrupted by one or more non-coding sections, which are denoted as introns. During the transcription of these genes a primary transcript is produced, the so-called pre-mRNA. After transcription, the introns are removed from the pre-mRNA and the non-coding sequences, the so-called exons, are joined together. This process is called pre-mRNA splicing.

The splicing out of introns takes place in the cell nucleus and results in the production of mature mRNA, which is exported from the cell nucleus into the cytoplasm and is used for translation. For the splicing of pre-mRNA the eukaryotic cell is equipped with a ribonucleoprotein complex, comprised of different proteins and five small RNA molecules, the so-called snRNAs (small nuclear RNAs). The proteins and snRNAs form small ribonucleoprotein particles (snRNPs) that provide for the recognition and the splicing out of the introns, thereby binding short conservative sequence sections of the pre-mRNA. These sequences are located within the intron at the border to the respective exon and are designated as 5′- or 3′-splice sites depending on the orientation in relation to the 5′- or 3′-end. In higher eukaryotes only the first and last two nucleotides of the 5′- and the 3′-splice site of the intron are conserved. In class I introns the dinucleotide GT is located at the 5′ splice site, the dinucleotide AG at the 3′ splicing side of the intron. In the less frequent class II introns the GT dinucleotide is replaced by an AC dinucleotide, and the AG dinucleotide is replaced by an AC nucleotide. A further element recognized by snRNPs is a conserved adenosine nucleotide, functioning as branch point in the splicing reaction. The branch point is surrounded by the consensus sequence YNCURAC and is normally located about 20-40 nucleotides in front of the 3′ splice site. Class II introns further include a pyrimidine rich section in this region. Class II introns are lacking this section.

In contrast to eukaryotic genes, prokaryotic genes normally have no intron-exon structure. They can, however, be organized in so-called operons, in which several genes are combined to a jointly regulated functional unit.

It would be desirable to have a possibility to produce or to let the cell produce eukaryotic or prokaryotic genes in vivo, if required, depending on the presence or absence of an appropriate signal external or internal to the cell, where required. Such a possibility is presently not known in the prior art. The object of the present invention is therefore to remedy this drawback.

The problem is solved by the subject matters of the independent claims.

The present invention provides a synthetic nucleic acid comprising at least one gene, containing, in coded form, an input for a biomolecular finite automaton, the processing of the input by the biomolecular finite automaton resulting in the spontaneous self-assembly of the at least one gene.

Unless expressly stated otherwise, the terms used in this application have the usual meaning known to a person skilled in the art. Some of the terms used in the application are additionally specified in more detail below.

By a “nucleic acid” is meant a polymer which monomers are nucleotides. A nucleotide is a compound composed of a sugar moiety, a nitrogen-containing heterocyclic organic base (nucleotide base or nucleobase) and a phosphate group. The sugar moiety is normally a pentose, in the case of DNA desoxyribose, in the case of RNA ribose. The nucleotides are linked via the phosphate group by means of a phosphodiester bridge between the 3′ C atom of the sugar component of a nucleoside (compound of a nucleobase and sugar) and the 5′ C atom of the sugar component of the next nucleoside. Normally, the nucleobases are purines (R) and pyrimidines (Y). Examples of purines are guanine (G) and adenine (A), examples of pyrimidines are cytosine (C), thymine (T) and uracil (U).

By “synthetic nucleic acid” is meant a nucleic acid being of synthetic origin, i.e. naturally not occurring as such. In particular, this term means that the nucleic acid has a nucleotide sequence and/or structure that is not present in a naturally occurring organism. A “synthetic nucleic acid” in the sense of the present invention may exert the same function in a cell as a naturally occurring nucleic acid. A synthetic nucleic acid according to the invention can, for example, be organized like a eukaryotic gene or a prokaryotic operon and may comprise one or more naturally occurring genes, which are expressed in the cell like naturally occurring genes. A nucleic acid organized like a eukaryotic gene can, for example, include the coding sequence of a naturally occurring gene, which may, however, be distributed over exons in a manner, that does not occur naturally, or have a naturally not occurring intron/exon structure. The intron/exon structure (e.g. number and sequence of exons/introns) may, for example, be taken from one organism, whereas the coding sequence in the exons is derived from another organism. Thus, the term “synthetic nucleic acid” is also meant to encompass nucleic acids which comprise naturally occurring components (e.g. exons, introns, genes), the combination or structure of which, however, cannot be found in a naturally occurring nucleic acid.

By “nucleotide sequence” is meant the linear sequence of nucleotides. Such a sequence is usually and also in the present application, unless not otherwise expressly stated or readily apparent for a person skilled in the art, presented by a sequence of one-letter abbreviations representing the nucleotides in 5′-3′ direction (e.g. ACGT is a linear sequence of the nucleotides adenine, cytidine, guanine and thymine).

By “gene” is meant a DNA segment carrying the information for the synthesis of a peptide or protein or of a structural or functional RNA (e.g. tRNA). The term “gene” as used in the present application also encompasses the primary RNA transcript of the gene.

By “exon” is meant a nucleotide sequence of the primary messenger RNA transcript (pre-mRNA) of a gene leaving the cell nucleus as part of the messenger RNA (mRNA) molecule. In the pre-mRNA, adjacent exons are separated by so-called introns, which are removed from the pre-mRNA before leaving the cell nucleus. In contrast to introns, exons are thus part of the mature mRNA. Exons normally include the open reading frames (ORF) of a protein, i.e. the sections coding for a protein. Exons may, however, contain exclusively or in addition to the ORFs sequence sections that are not translated into an amino acid sequence. These untranslated regions (UTR) are located at the 5′ and/or 3′ of the transcript, if applicable. The term exon also encompasses the respective nucleotide sequence of the DNA coding the pre-mRNA.

By “intron” is meant a nucleotide sequence of the pre-mRNA of a gene, which does not leave the cell nucleus as part of the mRNA molecule, i.e. which is not part of the mature mRNA. The term intron also encompasses the respective nucleotide sequence of the DNA coding the pre-mRNA. Introns are non-coding sections of the DNA within a gene flanked by exons. Introns are spliced out of the prem-RNA, before it is discharged from the cell nucleus for translation. Introns have conserved structures (intron signals), which are used by the cell to recognize the introns. Introns of class I, for example, begin (seen from 5′ direction) with the nucleotides GT (GU in the respective pre-mRNA) and end with the nucleotides AG. The GT dinucleotide designates the 5′ splice site, the AG dinucleotide the 3′ splice site. In addition to the 5′ splice site and the 3′ splice site at the intron borders the introns have a highly conserved adenosine nucleotide that serves as branch point during the splicing reaction. The branch point is normally located about 20-40 nucleotides in front of the 3′ splice site. Most introns further possess a pyrimidine rich region that is located between the branch point and the 3′ splice site.

By a “non-coding nucleotide sequence” or a “non-coding sequence” as used herein is meant a nucleotide sequence, which is not translated into an amino acid sequence according to the genetic code. For example, it can be an intron sequence of a gene. It can, however, also be a sequence located outside a gene, for example between the operator of an operon and the first gene of the operon or between the genes of an operon.

By “sense strand” is meant the strand of a double-stranded DNA containing the information in coded form. The sense strand therefore contains the sequence corresponding to the transcribed mRNA (with the exception that the mRNA contains U instead of T).

By “antisense strand” is meant the counter-strand of a double-stranded DNA complementary to the sense strand.

By a “promoter” is meant a section on the DNA involved in binding the RNA polymerase at the initiation of the transcription. The promoter region is located upstream from the gene.

By an “operon” is meant a group of genes which transcription is jointly regulated. An operon forms a functional unit on the DNA and comprises a promoter, an operator and one or more (structural) genes.

An “operator” is a recognition site within the operon at which the positive or negative control of the genetic transcription occurs by binding of an appropriate regulator, for example a repressor.

By “wild-type” is meant a naturally occurring organism, a naturally occurring nucleic acid or another naturally occurring structure.

A “finite automaton” or “finite state automaton” (in German also called “Zustands-maschine”, state machine) is a model of an information processing system with inputs and outputs, if applicable, having a finite number of possible (internal) configurations, so-called “states”, accepting particular inputs from a finite set of input symbols, the input alphabet, and producing corresponding output words, if any. One state is defined as initial state. State changes (transitions) are described by means of transition rules assigning any pair of current state and input a consecutive state. Formally, a finite state automaton (FSA) is thus characterized by a finite set of states (S), an input alphabet, at least one transition rule, at least one initial state (IS) and a set of final states. Generally, deterministic and non-deterministic finite automata are distinguished. In case of a deterministic finite automaton, for any state there exists exactly one transition for each input. In this case, the transition rule is a function. In case of a non-deterministic automaton there can be none or even more than one transition for the possible input. In this case, the transition rule is a relation. If the transition rule is defined by transition probabilities, and initial and final state(s) are defined by probability distributions, one speaks of a “stochastic finite automaton”. By a finite automaton in the sense of the present invention is also meant a device functioning according to the principle of a finite automaton. In addition, also a system of components, for example nucleic acid molecules, interacting in a manner that the system operates according to the principle of a finite automaton, is encompassed by the term “finite automaton”. By “system” is meant a number of components and their functional and/or structural interaction.

By a “biomolecular finite automaton” is meant a finite automaton operating with the aid of biomolecules, for example nucleic acid molecules. In particular, the term means a finite automaton that accepts biomolecules as input and biologically active molecules as output.

Biomolecules including an input, an initial or final state or a transition rule in coded form are also referred to as “input molecule”, “initial state molecule”, “final state molecule”, “transition state molecule” or “output molecule”, as the case may be. On the basis of the description herein and his technical knowledge the person skilled in the art will readily recognize in which context the terms “input”, “initial state”, “final state”, “transition state”, “output”, “input molecule”, “initial state molecule”, “final state molecule”, “transition state molecule” or “output molecule” are used in each case. For example, the terms “input”, “initial state”, “final state”, “transition state” and “output” may encompass the terms “initial state molecule”, “final state molecule”, “transition state molecule” or “output molecule”.

By “annealing” of a nucleotide sequence to a nucleic acid is meant the hybridization of the nucleotide sequence with the nucleic acid. In particular, the term means that at least 50%, preferably at least 60%, especially preferred at least 80%, more preferably at least 90%, more preferably at least 95%, even more preferably at least 99% and most preferably 100% of the nucleotides of the nucleotide sequence form a Watson-Crick base pairing with complementary nucleotides of the nucleic acid. Preferably the hybridization occurs under circumstances prevailing in a living cell.

By a “sticker automaton” or a biomolecular finite automaton operating according to a “sticker model” is meant a finite automaton wherein sections of a polymeric biomolecule, e.g. oligonucleotides, anneal to a polymeric biomolecule, preferably a single-stranded nucleic acid. The annealing biomolecule sections are referred to as “stickers”. For example, complementary oligonucleotides may anneal to a single-stranded DNA. The biomolecule sections preferably have less than 300, more preferably less than 200, more preferably less than 150, more preferably less than 100, more preferably less than 80, more preferably less than 50, more preferably less than 40, and still more preferably less than 30 monomers, e.g. nucleotides.

The nucleic acid according to the invention comprises at least one gene, the assembly instruction of which is given by the finite automaton. “Comprises” in the sense of the present invention also includes that the nucleic acid may be identical to the gene. A corresponding gene is also referred to as computational gene below. In an alternative embodiment, in which several genes are organized in form of an operon characteristic for prokaryotes the term “computational operon” may also be used. Unless expressly noted otherwise or unless otherwise unambiguously derivable from the context the term “computational gene” is, however, used in the present invention in such a way that it shall encompass the term “computational operon”. The gene or operon may result from spontaneous self-assembly. This spontaneous self-assembly may occur in vitro, occurs, however, preferably in vivo.

The nucleic acid of the invention with the computational gene or computational operon is formed by an autonomous computational process, preferably in vivo, i.e. in a living cell. The formation of a nucleic acid of the invention results from spontaneous self-assembly during the autonomous computational process. The autonomous computational process is preferably specified by an autonomous finite automaton. Preferably, the self-assembly does not occur in any case, but under a specific condition or specific conditions. This condition or these conditions are preferably describable by a Boolean expression encoded, for example, by biomolecules, preferably nucleic acids.

With the aid of the present invention it is, for example, possible, to generate eukaryotic genes and prokaryotic genes or operons, but also any other double-stranded nucleic acids in vivo, if required. In addition, there is also the possibility of a cascaded application, i.e. the generation of one or more additional computational genes. The nucleic acid according to the invention can advantageously be employed in different fields, for example in the fields of medicine for the diagnosis and/or therapy of diseases, For example for the targeted release of agents at the target site, in the fields of biotechnology for the targeted manipulation of cellular activities, for the screening of new enzymatic activities, for the production of recombinant proteins, for the protection of cells (for example plant cells) against viruses etc.

In a preferred embodiment, the nucleic acid according to the invention comprises at least one nucleotide sequence encoding at least one transition rule for the biomolecular finite automaton.

More preferably the nucleic acid according to the invention further comprises a) at least one nucleotide sequence encoding a symbol of an input alphabet for the biomolecular finite automaton, and b) at least one nucleotide sequence encoding at least one state of the biomolecular finite automaton. The nucleic acid encoding the at least one state of the biomolecular finite automaton is preferably encompassed by a spacer nucleotide sequence (“spacer”) and preferably forms a spacer nucleotide sequence, respectively.

In a preferred embodiment the nucleic acid of the invention comprises at least one non-coding sequence, wherein the nucleotide sequence encoding the symbol, the nucleotide sequence encoding the at least one state and the nucleotide sequence encoding the transition rule preferably are contained in the non-coding sequence. The non-coding sequence can, for example, be an intron of a gene, or a non-coding section of an operon.

In another preferred embodiment of the nucleic acid of the invention the non-coding sequence comprises an alternating series of nucleotide sequences encoding states and symbols, the series beginning and ending with a nucleotide sequence encoding a state.

More preferably the non-coding sequence is an intron of the at least one gene. In this embodiment the computational gene contains, analogous to naturally occurring eukaryotic genes, at least one intron and at least two exons. In contrast to a naturally occurring gene, however, the computational gene comprises a transition rule for the biomolecular finite automaton contained in the at least one intron, and preferably also symbols and states for the biomolecular finite automaton in coded form. In an embodiment of the computational gene with two exons the intron is preceded by an exon in the direction of the 5′ end of the nucleic acid and followed by an exon in the direction of the 3′ end of the nucleic acid. The computational gene may, however, also contain several introns and exons. Preferably the transition rule(s) and the symbols and states are located in the intron located to the 5′ end of the nucleic acid, but can also be included in another intron.

A computational gene may, for example, encode a eukaryotic wild-type protein in its exons. The corresponding naturally occurring gene of the wild-type protein then provides the function of the computational gene und is therefore called “functional gene”. The model for the construction of the computational gene, for example regarding intron-exon structure, number of exons and introns, conserved intron signals, location of start and stop codons, kind and location of promoters etc. can also be taken from the gene of the wild-type protein, but can also be taken from another naturally occurring gene, or can be completely synthetic. A gene whose basic structure serves as a model for a computational gene is called a “framework gene”, because, in a way, it provides the framework of the computational gene, whereas the function which the computational gene or its product fulfills or is intended to fulfill, stems from the “functional gene”. Although a computational gene can thus have the same function in a living cell than a naturally occurring gene (wild-type gene), it can differ therefrom in respect of its construction, for example regarding the location, number and length of introns and exons. The difference may also consist in a replacement of codons by synonymous codons.

In another preferred embodiment the computational gene is preceded by a promoter, which preferably, together with the exon located in the direction of the 5′ end of the nucleic acid and a 5′ splice site, defines the initial state of the biomolecular finite automaton. The promoter may be any promoter of natural or synthetic origin. The promoter is advantageously selected under consideration of the purpose the computational gene is intended to serve. For a computational gene that is intended to be expressed in a plant cell it is for example convenient to use a plant promoter that is able to exert its function in the target plant or target tissue.

In another preferred embodiment a section of the nucleic acid defines a final state of the biomolecular finite automaton, the final state comprising a branch site with an adenine nucleotide located within the intron, a 3′ splice site of the intron and the exon located in the direction of the 3′ end of the nucleic acid. More preferably the final state additionally comprises a pyrimidine-rich region located in 5′ direction behind the branch site.

In a preferred embodiment the at least one transition rule for the biomolecular finite automaton is encoded by a nucleotide sequence within the strand complementary to the sense strand of the computational gene.

More preferably, the sense strand of the gene with the preceding promoter sequence comprises the input for the biomolecular finite automaton.

Alternatively the nucleic acid of the invention may also comprise several genes arranged in the form of an operon. The operon comprises an operator, and the non-coding sequence is situated between that gene of the operon located closest to the 5′ end of the nucleic acid and the operator. In this manner, the nucleic acid is designed in the form of a prokaryotic operon that can be produced by spontaneous self-assembly, for example in a cell or in a reaction tube. Analogous to the description given above for a computational gene having a eukaryotic gene structure regarding “framework gene” and “functional gene” the “framework” of a computational gene having a prokaryotic operon structure may also be derived from a naturally occurring or synthetic operon. By a “framework operon” is meant a structure that is recognized and treated as an operon in a prokaryotic cell. The “functional genes” of such a “framework operon” may be wild-type genes, naturally occurring genes from another organism or also synthetic genes. In this manner, a computational gene may be provided with different functional genes, as required, whereas the framework of the computational operon, that is the basic structure making the computational operon recognizable in a prokaryotic cell, may remain the same.

The operon preferably comprises a promoter preferably encoding the initial state of the biomolecular finite automaton together with the operator. The final state of the biomolecular finite automaton preferably comprises the genes of the operon.

In a preferred embodiment of this alternative embodiment of the nucleic acid of the invention the at least one transition rule for the biomolecular finite automaton is encoded by a nucleotide sequence in the antisense strand. Preferably, it is also the case here that the transition rule(s) is(are) complementary to a non-coding section of the sense strand, the transition rule(s) may, however, also be complementary to a coding section of the sense strand.

Preferably, the sense strand with the preceding promoter sequence and the operator sequence comprises the input.

In a preferred embodiment, the nucleic acid of the invention may serve as a medicament. For example, the computational gene may assume the function of a natural gene mutated in a person to be treated. The computational gene can be formed by self-assembly via an autonomous computational process in the cell, and the self-assembly may occur under the condition that a mutation is present in the corresponding natural gene.

The invention also relates to a programmable biomolecular finite automaton with a finite set of states, at least one initial and at least one final state, the automaton being able make a transition from one state to another by at least one transition rule, and processing an input comprising at least one symbol of an input alphabet, the input being encoded in a nucleic acid comprising at least one gene.

The finite automaton of the invention processes biomolecules in form of nucleic acid molecules as input. Preferably the input or input molecule is a single-stranded nucleic acid molecule, for example a single-stranded DNA.

Preferably, the at least one transition rule is encoded by a nucleotide sequence encompassed by a non-coding sequence. The at least one transition rule is preferably single-stranded and complementary to sections of the sense strand of the non-coding sequence of the gene. The sections preferably comprise a nucleotide sequence encoding a symbol from the input alphabet and parts of spacer nucleotide sequences adjacent on both sides. In a preferred embodiment the spacer nucleotide sequences encode the states of the biomolecular finite automaton except the initial and final state.

In a preferred embodiment of the programmable biomolecular finite automaton the non-coding sequence is an intron of a gene. Alternatively, the non-coding sequence can be a section of an operon comprising several genes.

The invention also relates to a method for manufacturing a nucleic acid comprising at least one gene, wherein the nucleic acid is formed by self-assembly resulting from a computational process carried out by a biomolecular finite automaton. By means of the method a nucleic acid of the invention can be produced with a computational gene or computational operon in an autonomic manner. Autonomic means here that after the beginning of the computational process no external intervention is necessary.

In a preferred embodiment the computational process in the method of the invention comprises the processing of an input contained, in coded form, in the nucleic acid. Preferably, a single-stranded nucleic acid is used as input.

In the method of the invention, it is preferred to use an input comprising at least one nucleotide sequence comprising at least one nucleotide sequence encoding a symbol from an input alphabet of the biomolecular finite automaton.

More preferably, the nucleic acid comprises at least one non-coding sequence, wherein the transition rules of the biomolecular finite automaton are preferably encoded by nucleotide sequences encompassed by the non-coding sequence.

In an especially preferred embodiment of the method the non-coding sequence is an intron of a gene.

In a preferred embodiment of the method a single-stranded nucleic acid is used as input, preferably comprising at least one spacer nucleotide sequence comprising at least one nucleotide sequence encoding a symbol from an input alphabet of the biomolecular finite automaton, wherein the finite automaton is put into the initial state in that a single-stranded nucleotide sequence being complementary to a promoter sequence encompassed by the nucleic acid, to the exon following the promoter and to the 5′ splice site anneals to the nucleic acid, and wherein the finite automaton is going through further states by stepwise annealing, to the nucleic acid, of single-stranded nucleotide sequences encoding the transition rules and being complementary to intron sections, and reaches a final state in that a nucleotide sequence anneals to the nucleic acid comprising a nucleotide sequence complementary to the branch point of the intron, to the 3′ splice site of the intron and to the further exon or exons.

In an alternative embodiment the non-coding sequence is a section of an operon comprising several genes and an operator.

In this embodiment of the method of the invention a single-stranded nucleic acid is used as an input, preferably comprising at least one spacer nucleotide sequence comprising at least one nucleotide sequence encoding a symbol from an input alphabet of the biomolecular finite automaton, the finite automaton being put in the initial state by annealing of a single-stranded nucleotide sequence complementary to a promoter sequence encompassed by the nucleic acid and the operator sequence, the finite automaton going through further states by stepwise annealing of single-stranded nucleotide sequences encoding the transition rules und being complementary to sections of the non-coding sequences to the nucleic acid, and reaching a final state in that a nucleotide sequence is annealed to the nucleic acid that comprises a nucleotide sequence comprising the antisense strand to the genes of the operon.

In another preferred embodiment an accepted input results in a double-stranded DNA molecule comprising at least one gene that can be expressed in vivo, i.e. in a living cell, or in vitro, e.g. in a cell-free system.

In an especially preferred embodiment the method is carried out in a living cell. No protection is, however, claimed for carrying out the method for the purpose of a therapeutic treatment of the human or animal body and for the purpose of a diagnosis practiced on the human or animal body.

The invention also relates to a composition, comprising
a) a single-stranded nucleic acid containing an input for a biomolecular finite automaton in coded form,
b) a set of single-stranded nucleic acids complementary to sections of the single-stranded nucleic acid encoding the input, and containing transition rules of the biomolecular finite automaton in coded form
c) a single-stranded nucleic acid complementary to a section located at the 5′ end of the single-stranded nucleic acid encoding the input, and containing an initial state of the biomolecular finite automaton in coded form, and
d) a single-stranded nucleic acid complementary to a section located at the 3′ end of the single-stranded nucleic acid encoding the input, and containing a final state of the biomolecular finite automaton in coded form.

Like the nucleic acid of the invention, the composition of the invention is also suitable for use as medicament.

The ingredients of the composition may be present together, e.g. in a solution, preferably an aqueous solution, or separately, for example each in its own container.

Further, the invention relates to the use of a nucleic acid of the invention or a composition of the invention for the manufacture of a medicament or an intermediate product for a medicament, respectively.

The present invention is described in further detail below by means of illustrating examples and with reference to the accompanying figures.

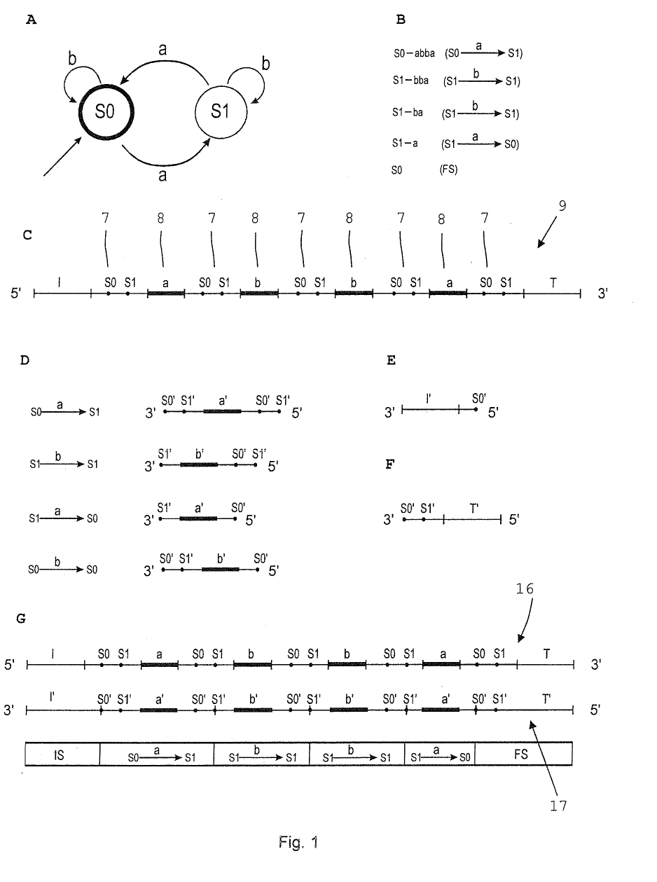

FIG. 1 illustrates an embodiment of the invention according to the complex “sticker” model. A. State diagram or state transition diagram of a finite automaton with an input alphabet {a, b} and set of states {S0, S1}. S0 is the initial and final state. S1=state 1. B. Computation for the input word “abba”. C. Encoding of the input “abba” by a single-stranded DNA molecule. D. Encoding of the transition rules. E. Encoding of the initial state. F. Encoding of the final state. G. Illustration of the accepted input word “abba” as double-stranded DNA molecule. IS=initial state, FS=final state, T=terminator, I=initiator. Nucleotide sequences complementary to the input are designated with an apostrophe (').

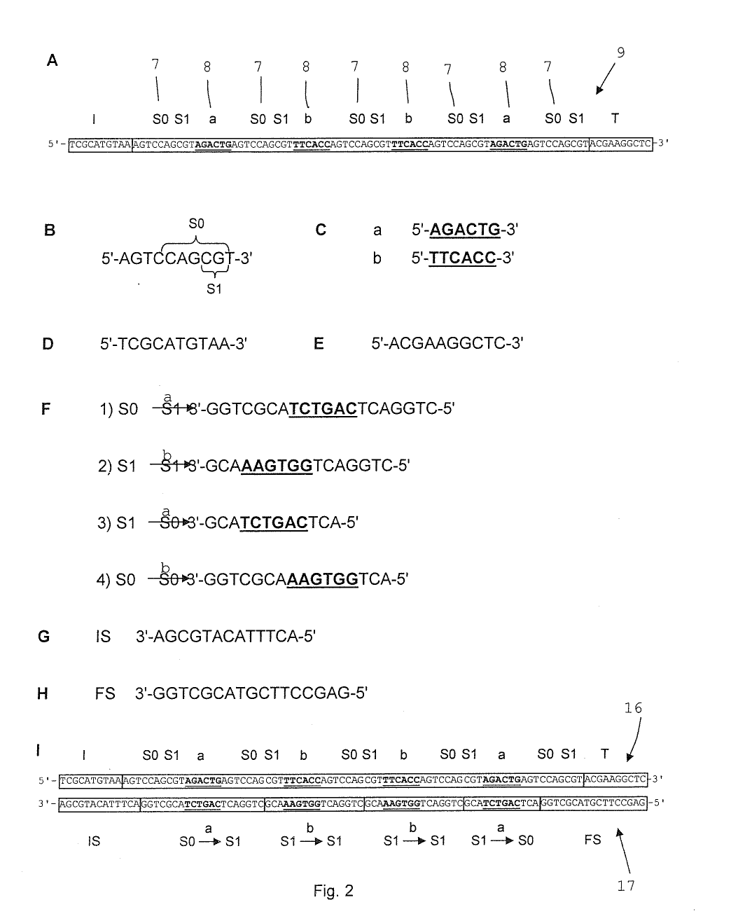

FIG. 2 shows an example for the realization of the finite automaton of FIG. 1 by means of nucleic acids. A. Input molecule, B. Spacer nucleotide sequence (spacer), C. Symbols a and b, D. Initiator I, E. Terminator T, F. Transition rules, G. Initial state IS, H. Final state FS, I. Double-stranded DNA as a result of an accepted input.

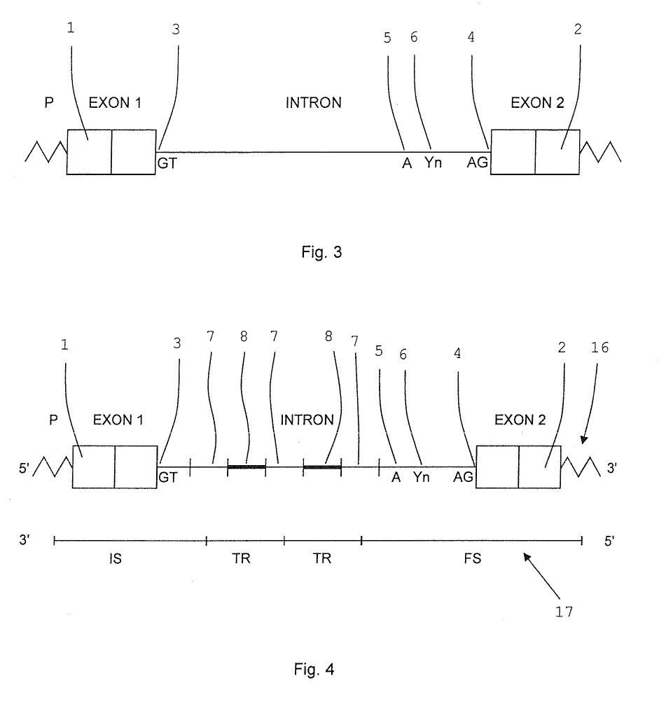

FIG. 3 shows a schematic illustration of a eukaryotic gene with two exons (exon 1 and exon 2) separated by an intron. The dinucleotide GT denotes the 5′ splice site, the dinucleotide AG the 3′ splice site, A the branch point and Yn the pyrimidine-rich region (Y=pyrimidine, n=number of pyrimidine nucleotides, about 6-17). P=promoter.

FIG. 4 shows a schematic illustration of an embodiment of the nucleic acid of the invention with a “computational gene”. P=promoter, IS=Initial state, TR=transition rule, FS=final state.

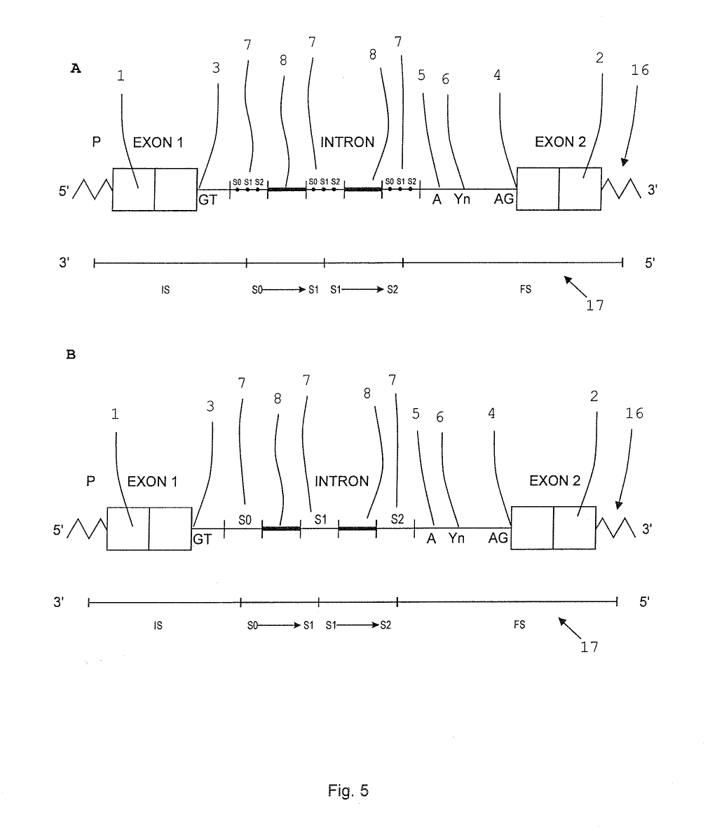

FIG. 5 shows two preferred embodiments of the invention. FIG. 5A illustrates an embodiment according to a complex “sticker” model, FIG. 5B illustrates an embodiment according to a simple “sticker” model. P=promoter, IS=initial state, FS=final state.

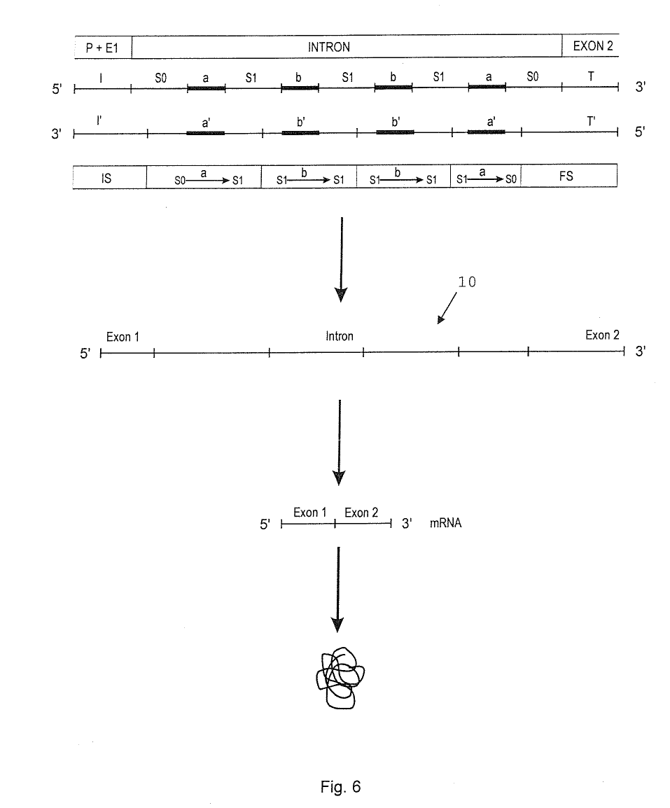

FIG. 6 shows the expression of a computational gene. An accepted input provides a complete double-stranded DNA molecule that can be expressed in the cell by spontaneous self-assembly. P=promoter, E1=Exon 1, I=Initiator, T=Terminator, IS=Initial state, FS=Final state.

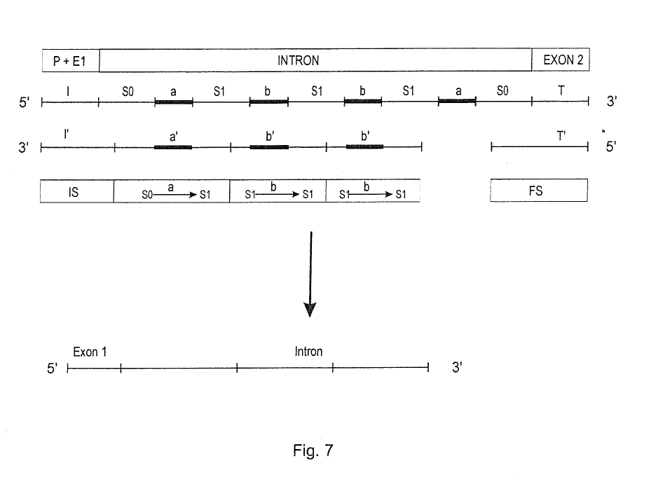

FIG. 7 shows the case of a non-accepted input. The only partially double-stranded DNA molecule is not expressed in the cell. P=promoter, E1=Exon 1, IS=Initial state, FS=final state.

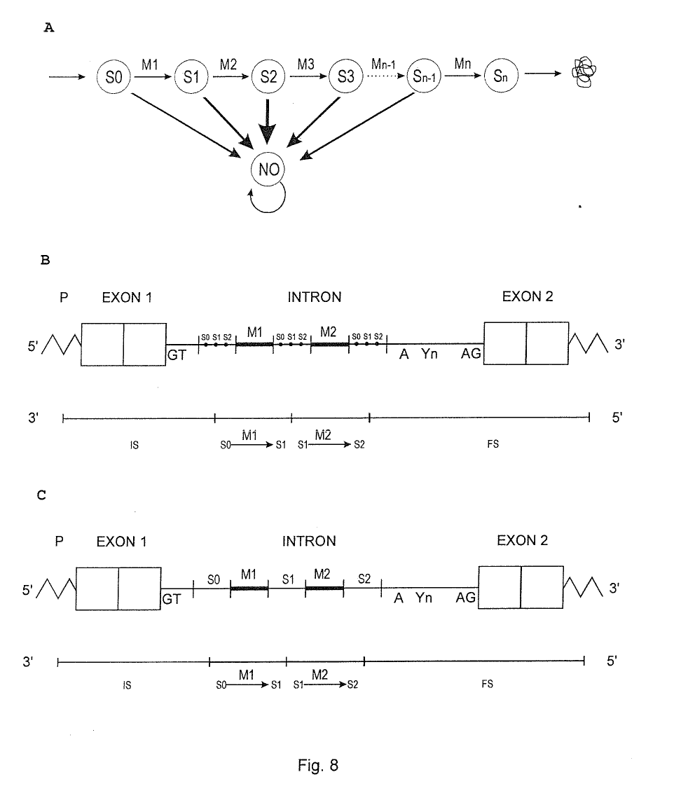

FIG. 8 shows the implementation of “diagnostic rule”. A. Finite automaton for the diagnostic rule. B. Corresponding computational gene synthesized according to the complex sticker model. C. Corresponding computational gene synthesized according to the simple sticker model. M=Mutation; NO=No.

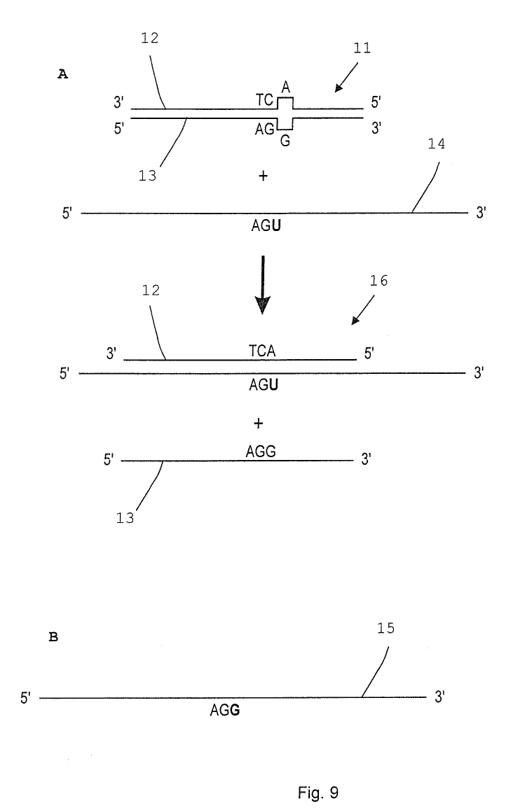

FIG. 9A exemplifies a scheme for the detection of a mutation at molecular level.

FIG. 9B depicts the corresponding non-mutated mRNA.

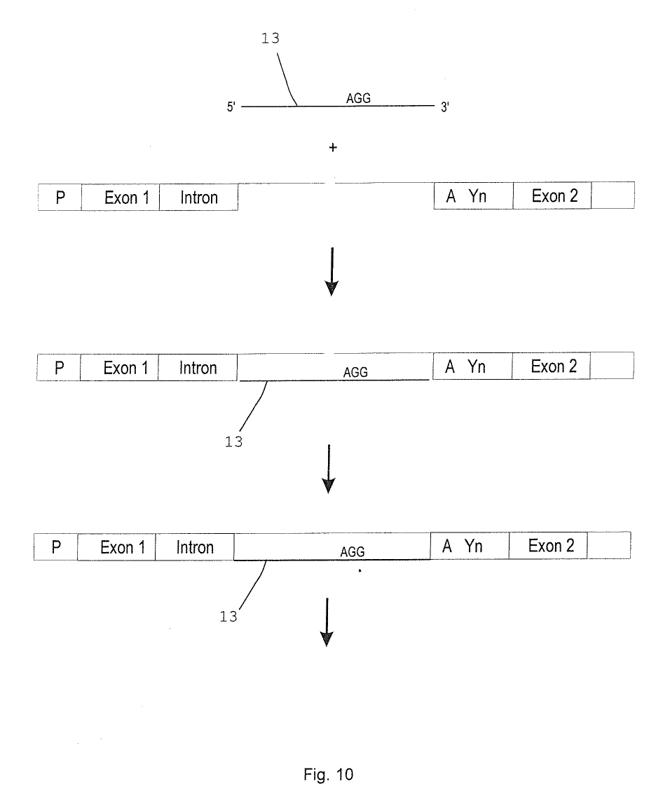

FIG. 10 schematically shows a preferred embodiment of the process of the invention. P=promoter.

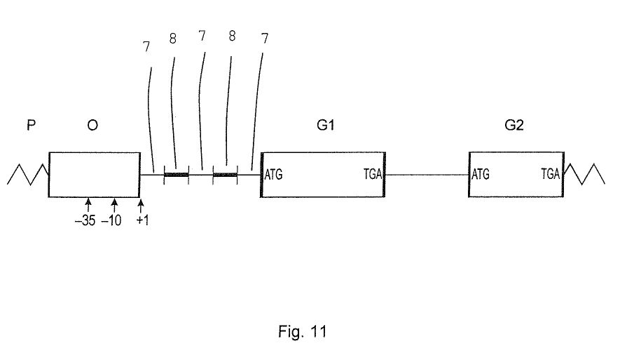

FIG. 11 schematically shows a computational operon of the invention. P=promoter, O=operator, G=gene.

### Example 1

FIG. 1 schematically shows components of a biomolecular finite automaton operating according to a preferred mechanism called “complex sticker model” below. The present invention is, however, not restricted to this model. Other models, e.g. simpler “sticker models” can also be employed in connection with the present invention. An automaton operating according to a sticker model is also briefly called “sticker automaton”.

The biomolecular finite automaton illustrated in FIG. 1 can be in two states, S0 and S1, and process two symbols. There is, however, no limitation to this number of states and symbols. In contrast, the Shapiro model described above is limited to two states and two symbols, strongly limiting the complexity (specified as product of the number of symbols and the number of states). The “complex” sticker model described herein can code as many states and symbols as necessary. Further, it should be noted, that the sticker model can be expanded to stochastic finite automata in the same manner as the Shapiro model.

In FIG. 1A a corresponding state diagram is depicted. Circles symbolize a state that the automaton can accept. The (accepted) final state is indicated by the circle given in heavy print. Here, the initial state IS corresponds to the state S0, and the initial state is identical to the final state (FS). Arrows in the diagram indicate the state transitions. Above the arrow, symbols are depicted that can be processed by the automaton (here the symbols a and b) and at the processing of which the state transition occurs. The straight arrow denotes the entry of the automaton into the initial state S0. The depicted automaton accepts inputs with an even number of the symbol “a”.

In FIG. 1B the processing of input word “abba” is depicted. The automaton initially is in the initial state S0 and processes the first symbol “a” from the input word, leading to a transition to the state S1. The successive processing of the two following “b” symbols does not lead to an apparent state change; instead the automaton makes a transition from state S1 to state S1 again. The input of the last symbol “a” leads to a transition to the state S0, which at the same time is the final state FS. An input is considered “accepted” if the automaton, after its processing, is in a final state envisaged as such.

In FIG. 1C a single-stranded DNA molecule 9 is shown forming the input of the sticker automaton. In the sticker model, unlike with the Shapiro model, the input is encoded by a single-stranded nucleic acid or DNA (ssDNA). In addition, the input molecule is not digested, in contrast to the Shapiro model. The single-stranded DNA comprises the initiator 1, an alternating series of spacer nucleotide sequences (“spacer”) 7 encoding states S and nucleotide sequences 8 (in short: symbol sequences) encoding symbols as well as the terminator T.

In FIG. 1D transition rules TR encoded by single-stranded nucleic acids, e.g. ssDNA, are shown. The single-stranded nucleic acids are complementary to sections on the input molecule 9, so that they anneal to these sections, i.e. can hybridize with these sections. Since the single-stranded nucleic acids practically stick to the input molecule, they are also called “stickers”. Transition rules have the structure:

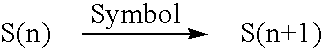

S(n) corresponds to the respective current state, S(n+1) to the respective next state. The transition rules are encoded by single-stranded nucleic acids (oligonucleotides), which are complementary to the 5′-S(n)-part of the spacer nucleotide sequence 7, the symbol and the 3′-S(n+1)-part of the spacer nucleotide sequence 7. In the Figure, the four transition rules predefined in this example are shown:

1. Transition from S0 to S1 under processing of symbol “a”

2. Transition from S1 to S1 under processing of symbol “b”

3. Transition from S1 to S0 under processing of symbol “a”

4. Transition from S0 to S0 under processing of symbol “b”

The four additional transition rules possible in the two-state-two-symbols-automaton described herein are not depicted.

By selecting or predefining the corresponding transition rules from the group of possible transition rules, encoded in single-stranded nucleic acids, the biomolecular finite automaton can be programmed.

In FIG. 1E and 1F the initial state IS and the final state FS are encoded. Again, these are single-stranded nucleic acids complementary to specific sections of the input molecule 9. The nucleic acid encoding the initial state IS is complementary to the initiator sequence and the 5′-part of the section of the following “spacer” sequence S0 encoding the initial state IS (here S0). Annealing of this single-stranded nucleic acid puts the automaton in the initial state S0. The nucleic acid encoding the final state FS is complementary to the terminator sequence und part of the “spacer” 7, located, in 5′ direction, in front of it, encoding the final state FS (here also S0).

In FIG. 1G a double-stranded DNA (dsDNA) is shown, being the result of the accepted input of the input word “abba”. In case of an accepted input, the computational process results in a complete double-stranded DNA, since all complementary nucleic acids (“stickers”) are annealed to the input molecule in a manner, that no gap is remaining in the complementary strand. Partially incomplete DNA can be digested by means of nucleases, which are well known to the person skilled in the art. In vitro, for example, the mung bean nuclease or S1 nuclease may be used, the S1 nuclease being preferred. If the computational process is performed within the cell, cellular enzymes carry out the digestion.

In FIG. 2 it is exemplary shown how the finite automaton depicted schematically in FIG. 1 may be realized with nucleic acids. FIG. 2A shows the input molecule 9, a single-stranded DNA molecule. The input molecule 9 comprises the initiator I (see FIG. 2D), an alternating series of spacer nucleotide sequences 7 encoding the two states S0 and S1 (see FIG. 2B) and symbol sequences 8 (see FIG. 2C) as well as the terminator T (see FIG. 2E). The symbol sequences 8 are marked by bold print and additional underline.

FIG. 2F shows the four transition rules selected from a total number of eight possible transition rules TR. The oligonucleotides encoding the transition rules TR are depicted in 3′-5′ direction and are complementary to the start and end section, respectively, of the input molecule 9.

FIG. 2I shows the result of an input accepted by the finite automaton. During the computational process, the oligonucleotides encoding the final IS, the transition rules TR and the final state FS have annealed in the correct order, resulting in a double-stranded DNA molecule. During the computational process the finite automaton was put into the initial state IS=S0 by annealing of the nucleotide sequence depicted in FIG. 2G. As can be seen from FIG. 2B, the nucleotide sequence CCAGCGT in the corresponding spacer nucleotide sequence is freely accessible, i.e. not covered by complementary bases, whereas the preceding sequence AGT is covered by the spacer nucleotide sequence 7 by means of base pairing. Subsequently, the automaton made a transition from the state S0 into the state S1 by annealing of the transition rule nucleotide sequence depicted in FIG. 2F 1) under processing of the symbol a. This state is identifiable in that the sequence CCAG in the corresponding spacer nucleotide sequence 7 is covered by base pairing, whereas the sequence CGT is freely accessible. By annealing the transition rule nucleotide sequence depicted in FIG. 2F 2) twice the automaton switched again from state S1 to state S1, under respective processing of the symbol b. By annealing the transition rule nucleotide sequence depicted in FIG. 2F 3) the automaton made a transition from state S1 to state S0, being also the final state, under respective processing of the symbol a. Finally, the nucleotide sequence depicted in FIG. 2H anneals to the input molecule 9. A further change of state is, however, not associated therewith. The automaton is still in state S0.

### Example 2

FIG. 3 schematically shows the structure of a typical eukaryotic gene with a class-I-intron. The intron is flanked by two exons. A promoter P precedes the first exon. The first exon contains at the 5′ end a non-translated region (5′ UTR) 1, the second exon contains at the 3′ end a non-translated region (3′ UTR) 2. The intron contains a 5′ splice site 3 at its flank to the 5′ end, and a 3′ splice site 4 at its flank to the 3′ end. In addition, the intron contains a branch point 5 and a pyrimidine rich region 6 between the branch point and the 3′ splice site.

FIG. 4 schematically illustrates an embodiment of a computational gene according to the present invention, having a structure analogous to the eukaryotic gene shown in FIG. 3. The computational gene is preceded by a promoter P. As “framework” the computational gene contains two exons and one intron, the intron comprising the intron signals of a class I intron, i.e. the 5′ and 3′ splice sites 3, 4, the branch point 5 and the pyrimidine-rich region 6. The intron also comprises the spacer nucleotide sequences 7 and nucleotide sequences 8 (“spacer”) encoding symbols from an input alphabet for the biomolecular finite automaton. The spacer nucleotide sequences 7 and the nucleotide sequences 8 encoding symbols are arranged in an alternating series, the series beginning and ending with at least one spacer nucleotide sequence 7 encoding a state. The spacer nucleotide sequence 7 and the nucleotide sequences 8 encoding symbols are arranged on the sense strand 16 of the computational gene. The strand 17 (antisense strand) complementary to the sense strand 16 comprises nucleotide sequences coding for the initial state IS, the transition rules TR and the final state FS of the biomolecular finite automaton. The initial state IS here comprises the promoter P and the first exon (which, together, form the “initiator”), the 5′ splice site 3 and the 5′-part of the first spacer nucleotide sequence 7 following the 5′ splice site. The transition rules TR comprise nucleotide sequences that are complementary to the 5′-part of a spacer nucleotide sequence 7 encoding the current state of the automaton, to a nucleic acid sequence 8 encoding a symbol and the 3′-part of a spacer nucleotide sequence 7 encoding the next state of the automaton. The final state FS comprises the 3′-part of the last spacer nucleotide sequence 7 located in front of the 3′ splice site.

FIG. 5A shows a preferred embodiment of the present invention, in which the self-assembly occurs according to the complex sticker model. In this example, each of the spacer nucleotide sequences 7 encodes states S0, S1 and S2.

In FIG. 5B a further embodiment is shown operating according to a simple “sticker” model. Here too, more than two states are possible, however, one spacer nucleotide sequence 7 only encodes one state S at a time.

FIG. 6 shows an example for a computational process by means of a biomolecular finite sticker automaton of the invention, in which the input contained in a single-stranded nucleic acid in coded form was accepted. The double-stranded DNA, comprising an artificial gene with a preceding promoter P, resulting from the autonomously performed computational process, can be expressed in a cell like a naturally occurring gene. The transcription of the gene leads to a pre-mRNA 10. In a further step, the splicing process leads to an mRNA which may, for example, be translated into a protein, or which also may assume another function.

FIG. 7 shows the result of a non-accepted input. The autonomous computational process results in the formation of a partially double-stranded DNA, which will not be translated within the cell.

### Example 3

In the following, on the basis of an example from the fields of medicine, the possibilities opening up with the aid of the present invention shall be illustrated. It will be apparent for the skilled person that the invention can easily be applied to other fields outside medicine. In particular, the invention can advantageously be applied in the fields of biotechnology, for example plant biotechnology.

Computational genes may, for example, be used to develop a treatment mechanism for aberrated genes. Aberrated genes are mainly induced by gene mutation. Gene mutations form spontaneously, i.e. without external influence, or are induced by chemicals or radiation. The mechanisms of the spontaneous or induced triggering of mutations (mutagenesis) are diverse, but they have the same consequences. The most important types of intragenic mutations are neutral, nonsense and missense mutations. Neutral mutations do not alter the genetic information. A codon is simply converted to a synonymous codon. Nonsense mutations convert sense codons into stop codons. In this case an incomplete protein fragment is synthesized, normally resulting in a loss of the function of the original protein. In contrast, missense mutations alter the genetic information and may have different consequences, depending on type and location of the amino acid being replaced in the protein. In the worst case the cell may perish or become a tumor cell. Many types of human cancers are, for example, caused by specific missense mutations in tumor suppressor or oncogenes (Hainaut, P. and Hollstein, M.: Adv. Cancer Red., 77, 81-137, 2000).

Today, different treatment strategies for different classes of oncogenic mutations are provided for. With the aid of computational genes, a novel, more general treatment mechanism can be developed. This mechanism is based on a rule, which, in the fields of medicine, may be called a diagnostic rule. The diagnostic rule allows for a molecular diagnosis of diseases and is defined by a Boolean expression B in one or more variables. The Boolean variables are given by molecular markers which are either present (value true) or absent (value false). The term “molecular marker” primarily comprises gene mutations, but also an altered gene expression level or an altered protein structure.

A typical Boolean expression has the form

B=mol_marker_{—}1 and mol_marker_{—}2 and . . . and mol_marker_n (1)

A typical diagnostic rule has the following form:

If B then produce (computational gene) (2)

In case of a positive diagnosis an aberrated gene is present in the cell. Thereupon, a corresponding computational gene is produced. In addition the aberrated gene may be switched off. The computational gene produced may, for example, encode the protein of the wild-type gene corresponding to the aberrated gene or a peptide as counteragent. In the first case, the function of the aberrated gene is restored. The switch-off of the aberrated gene may be accomplished by the release of a short antisense nucleic acid that binds to the mRNA of the aberrated gene thus preventing its translation. This rescue mechanism controls the gene expression in a logical manner and permits the implementation of complex rules for the molecular diagnosis and therapy of diseases. The mechanism is universally applicable to any disease detectable by means of suitable molecular markers.

The computational gene is generated in vivo by an autonomous computational process, whose input is represented by or is contained in the molecular markers from the respective diagnostic rule. FIG. 8 shows the implementation of the diagnostic rule by means of a sticker automaton. The symbols of the finite automaton are formed by molecular markers (here mutations M). If all molecular markers are processed by the finite automaton, this means a positive diagnosis and the simultaneous spontaneous self-assembly of a corresponding computational gene, which, for example, encodes a non-mutated wild-type protein, generated as a result of the autonomous computational process.

In the following, the treatment strategy presented above is described in more detail on the basis of colon cancer as an example. It is known that a point mutation of the p53 protein in codon 249 may cause colon cancer (Montesano, R., Hainaut, P, and Wild, C. P.: Hepatocellular carcinoma: From gene to public health. J. Natl. Cancer Inst., 89, 1844-1851, 1997). The corresponding diagnostic rule is as follows:

If p53_mutaded_at_Codon_{—}129 then produce (healthy_p53_or/and_CDB3) (3)

The p53 protein is a tumor suppressor. In more than 50 percent of the human cancer diseases missense mutations are present in p53, which are to be found predominantly in subunit p53C. These mutations are grouped in two classes: DNA contact mutations reducing the number of DNA binding residues, and structural mutations resulting in a conformational change of p53C (Cho, Y, Gorina, S., Jeffrey, P. D. and Pavietich, N. P: Science, 265, 346-355, 1994). The CDB3 peptide can bind to the subunit p53C and thus stabilize its structure. Therefore, CDB3 can be used as a rescue mechanism in case of structural mutations of p53C, whereas other strategies are necessary for DNA contact mutations.

In order to interpret the Boolean expression in (3) point mutations must be detected (see FIG. 9). For this purpose, a so-called diagnostic complex 11 is used. This is a double-stranded nucleic acid molecule, preferably a DNA molecule, consisting of a mutation signal 12 and a diagnostic signal 13. Both signals are antiparallel and complementary, except for the mutated site. Thermodynamic studies (Bullock, A. N. and Fersht, A. R.: Nat. Cancer Rev., 1, 68-76, 2001; A. J. Turberfield, J. C. Mitchell, B. Yurke Jr., A. P. Mills, M. I. Blakey and F. C. Simmel: DNA fuel for free-running nanomachines. Phys. Rev. Lett., 90, 118102 pp, 2003) show that, in case of a positive diagnosis, the mutated mRNA 14 preferably forms a partially double-stranded DNA/RNA complex 16 with the mutation signal 12, releasing the diagnostic signal 13 (see FIG. 9A, FIG. 9B shows the non-mutated mRNA 15). The DNA/RNA complex is inactivated by DNAse H. The mutation signal 12 released from the diagnostic complex 11 acts as inhibitor preventing the expression of the mutated gene. The diagnostic signal 13 is a molecular marker serving as an input of the diagnostic rule (3). The signal provides a computational gene by spontaneous self-assembly (see FIG. 10). With the aid of this mechanism it is, for example, possible to generate one or more input molecule(s) for a biomolecular finite automaton and/or one or more transition rule molecule(s) for a biomolecular finite automaton in a cell.

Possibly, several sites have to be mutated and the length (i.e. the number of base pairs) of the diagnostic signal has to be increased, respectively, in order to enhance the efficiency of the process illustrated in FIG. 9A.

The computational gene in the diagnostic rule (3) encodes either a wild-type p53 or CDB3. For encoding these products human genes, e.g. with two exons, can be taken as frameworks, the genes being preferably expressed in all tissues, e.g. ID1 (Inhibitor of DNA Binding 1) or ADP ribosylation factor 6 (ARF6). For example, ID1 and ARF6, respectively, can be used to specify a computational gene for CDB3 and p53, respectively. From the framework gene, the corresponding computational gene adopts the conserved patterns.

### Example 4

In this example the self-assembly of a prokaryotic operon is described with reference to FIG. 11.

Prokaryotic genes are often organized in the form of operons. An operon represents a section on the DNA having a promoter, an operator and a series of genes. The genes may be structural genes. Promoter, operator and genes, respectively, are separated by non-coding regions. The expression of the series of genes in an operon can be switched on or off by particular substances taken up by the cell. In this manner, the protein biosynthesis is activated or inhibited. A repressor protein may, for example, be attributed to an operon, the repressor protein binding to the operator and preventing the RNA polymerase located at the promoter from transcribing the gene-coding sequence. For example, the repressor of the lactose operon changes its steric structure when the cell takes up lactose. Thus, the repressor is no longer able to bind to the operator. In this case, the RNA polymerase can jointly transcribe the genes of the operon. These genes synthesize enzymes for the lactose decomposition in the cell.

In bacterial cells, computational genes may also be synthesized by means of operons. The structure of an operon consisting of two genes is shown in FIG. 11. The non-coding region between the operator and the first gene is used for encoding states and symbols for the synthesis of the computational operon. The sticker nucleic acids of the states and transitions may, however, also be complementary to coding regions. It is, however, advantageous to encode a symbol or a state of the automaton by a DNA section of the coding region, because such a region can be synthesized independently, if it contains the start codon ATG. Such stickers are then not available for the spontaneous self-assembly of the computational operon.

The assembly instruction of the computational operon is given by the finite automaton. The computational gene results form spontaneous self-assembly. Each transition rule preferably consists of a region of the non-coding region between operator and the first gene following downstream. The initial state encodes the promoter and the operator. The final state comprises one or more genes including the separating non-coding regions located between the genes, if any.

1. A nucleic acid comprising at least one gene, wherein the nucleic acid contains, in coded form, an input for a biomolecular finite automaton, whose processing by the biomolecular finite automaton resulting in the spontaneous self-assembly of the at least one gene, and wherein the nucleic acid is a synthetic nucleic acid.

2. The nucleic acid according to claim 1, wherein the nucleic acid comprises at least one nucleotide sequence encoding at least on transition rule for the biomolecular finite automaton.

3. The nucleic acid according to claim 2, wherein the nucleic acid
a) comprises at least one nucleotide sequence encoding a symbol of an input alphabet for the biomolecular finite automaton, and
b) comprises at least one nucleotide sequence encoding at least one state of the biomolecular finite automaton.

4. The nucleic acid according to claim 3, wherein the nucleotide sequence encoding the symbol, the nucleotide sequence encoding the at least one state and the nucleotide sequence encoding the transition rule are contained in a non-coding sequence.

5. The nucleic acid according to claim 4, wherein the non-coding sequence comprises an alternating series of nucleotide sequences encoding states and symbols, the series beginning and ending with a nucleotide sequence encoding a state.

6. The nucleic acid according to claim 4, wherein the non-coding sequence is an intron in the gene, wherein the intron is preceded by an exon in the direction of the 5′ end of the nucleic acid and followed by an exon in the direction of the 3′ end of the nucleic acid.

7. The nucleic acid according to claim 6, wherein the exon located in the direction of the 5′ end of the nucleic acid, together with a 5′ splice site of the intron and a promoter preceding the gene, defines the initial state of the biomolecular finite automaton.

8. The nucleic acid according to claim 6, wherein the final state of the biomolecular finite automaton comprises a branch site with an adenine nucleotide located within the intron, a 3′ splice site of the intron and the exon located in the direction of the 3′ end of the nucleic acid.

9. The nucleic acid according to claim 8, wherein the final state additionally comprises a pyrimidine-rich region located in 5′ direction behind the branch site.

10. The nucleic acid according to claim 2, wherein the at least one transition rule for the biomolecular finite automaton is encoded by a nucleotide sequence within the strand complementary to the sense strand of the gene.

11. The nucleic acid according to claim 1, wherein the sense strand of the gene with a preceding promoter sequence comprises the input.

12. The nucleic acid according to claim 4, wherein the nucleic acid comprises an operon comprising one or more genes with an operator and that the non-coding sequence is located between the gene located in the direction of the 5′ end of the nucleic acid and the operator.

13. The nucleic acid according to claim 12, wherein the operon comprises a promoter which, together with the operator, defines the initial state of the biomolecular finite automaton.

14. The nucleic acid according to claim 12, wherein the final state of the biomolecular finite automaton comprises the genes of the operon.

15. The nucleic acid according to claim 12, wherein the at least one transition rule for the biomolecular finite automaton is encoded by a nucleotide sequence in the antisense strand.

16. The nucleic acid according to claim 12, wherein the sense strand with the preceding promoter sequence and the operator sequence comprises the input.

17. The A nucleic acid according to claim 1 for use as a medicament.

18. A programmable biomolecular finite automaton with a finite set of states, at least one initial and at least one final state, the automaton being able to make a transition from one state to another by at least one transition rule, and processing an input comprising at least one symbol of an input alphabet, wherein the input is encoded in a nucleic acid comprising at least one gene.

19. The programmable biomolecular finite automaton according to claim 18, wherein the input is a single-stranded DNA.

20. The programmable biomolecular finite automaton according to claim 18, wherein the at least one transition rule is encoded by a nucleotide sequence encompassed by a non-coding sequence.

21. The programmable biomolecular finite automaton according to claim 20, wherein the transition rule(s) is (are) encoded by (a) single-stranded nucleotide sequence(s) complementary to (a) section(s) of the non-coding sequence, the section(s) comprising a nucleotide sequence encoding a symbol of the input alphabet and parts of spacer nucleotide sequences adjacent on both sides.

22. The programmable biomolecular finite automaton according to claim 21, wherein the spacer nucleotide sequences encode the states of the biomolecular finite automaton except the initial and final state.

23. The programmable biomolecular finite automaton according to claim 20, wherein the non-coding sequence is an intron of a gene.

24. The programmable biomolecular finite automaton according to claim 18, wherein the non-coding sequence is a section of an operon comprising several genes.

25. A method for manufacturing a nucleic acid comprising at least one gene, wherein the nucleic acid is formed by self-assembly resulting from a computational process carried out by a biomolecular finite automaton.

26. The method according to claim 25, wherein the computational process comprises the processing of an input contained, in coded form, in a nucleic acid by a biomolecular finite automaton.

27. The method according to claim 26, wherein a single-stranded nucleic acid is used as input.

28. The method according to claim 27, wherein the input comprises at least one nucleotide sequence comprising at least one nucleotide sequence encoding a symbol of an input alphabet of the biomolecular finite automaton.

29. The method according to claim 25, wherein the nucleic acid comprises at least one non-coding sequence, and that the transition rules of the biomolecular finite automaton are encoded by nucleotide sequences encompassed by the non-coding sequence.

30. The method according to claim 29, wherein the non-coding sequence is an intron of a gene containing at least two exons.

31. The method according to claim 30, wherein as input a single-stranded nucleic acid is used comprising at least one spacer nucleotide sequence comprising at least one nucleotide sequence encoding a symbol of an input alphabet of the biomolecular finite automaton, the finite automaton being put into the initial state by annealing of a single-stranded nucleotide sequence complementary to a promoter sequence encompassed by the nucleic acid, to the exon following the promoter and the 5′ splice site to the nucleic acid, the finite automaton going through further states by stepwise annealing of single-stranded nucleotide sequences encoding the transition rules and being complementary to intron sections to the nucleic acid, and reaching a final state in that a nucleotide sequence is annealed to the nucleic acid comprising a nucleotide sequence complementary to the branch point of the intron, to the 3′ splice site of the intron and to the further exon(s).

32. The method according to claim 29, wherein the non-coding sequence is a section of an operon comprising several genes and an operator.

33. The method according to claim 32, wherein as input a single-stranded nucleic acid is used comprising at least one spacer nucleotide sequence comprising at least one nucleotide sequence encoding a symbol of an input alphabet of the biomolecular finite automaton, the finite automaton being put into the initial state by annealing of a single-stranded nucleotide sequence complementary to a promoter sequence encompassed by the nucleic acid and the operator sequence, the finite automaton going through further states by stepwise annealing of single-stranded nucleotide sequences encoding the transition rules und being complementary to sections of the non-coding sequence to the nucleic acid, and reaching a final state in that a nucleotide sequence is annealed to the nucleic acid comprising a nucleotide sequence comprising the antisense strand to the genes of the operon.

34. The method according to claim 25, wherein an accepted input results in a double-stranded DNA molecule comprising at least one gene that can be expressed in vivo or in vitro.

35. The method according to claim 25, wherein the method is carried out in a living cell, except for the purpose of the therapeutic treatment of the human or animal body and for the purpose of a diagnoses practiced on the human or animal body.

36. A composition, comprising
a) a single-stranded nucleic acid containing an input for a biomolecular finite automaton in coded form,
b) a set of single-stranded nucleic acids complementary to sections of the single-stranded nucleic acid encoding the input, and containing transition rules of the biomolecular finite automaton in coded form
c) a single-stranded nucleic acid complementary to a section located at the 5′ end of the single-stranded nucleic acid encoding the input, and containing an initial state of the biomolecular finite automaton in coded form, and
d) a single-stranded nucleic acid complementary to a section located at the 3′ end of the single-stranded nucleic acid encoding the input, and containing a final state of the biomolecular finite automaton in coded form.

37. The composition according to claim 36 for use as medicament.

38. Use of a nucleic acid according to claim 1 for the manufacture of a medicament or an intermediate product for a medicament.

39. Use of a composition according to claim 36 for the manufacture of a medicament or an intermediate product for a medicament.
